(19) United States
(12) Patent Application Publication
(10) Pub. No.: US 2010/0311986 A1
(43) Pub. Date: Dec. 9, 2010

(54) PREPARING METHOD OF METHOXYPOLYETHYLENEGLYCOL ETHYLMALEIMIDE

(75) Inventors: Pyeong-Uk Park, Dong-gu Gwangju (KR); Seong-Nyun Kim, Nam-gu Ulsan (KR); Su-Chan Kim, Jung-gu Ulsan (KR); Jung-Oh Kwon, Nam-gu Ulsan (KR)

Correspondence Address: CANTOR COLBURN LLP, 20 Church Street, 22nd Floor, Hartford, CT, 06103, US

(73) Assignee: ID BIOCHEM, INC., Seoul (KR)

(21) Appl. No.: 12/669,722

(22) PCT Filed: Mar. 18, 2008
(86) PCT No.: PCT/KR2008/001506

§ 371 Date: Jun. 14, 2010

(30) Foreign Application Priority Data
Jul. 18, 2007 (KR) 10-2007-0071653

(51) Int. Cl. C07D 207/40

(52) U.S. Cl. 548/547

(57) ABSTRACT

The present invention relates to a process for preparing methoxy polyethylene glycol ethylmaleimide (abbreviated to as “mPEG-ethylmaleimide”) and derivatives thereof with high purity.
methoxypolyethyleneglycol ethylmaleimide therefrom.

### TECHNICAL FIELD

The present invention relates to a process for preparing methoxy polyethylene glycol ethylmaleimide (hereinbelow, referred to as “mPEG-ethylmaleimide”) and derivatives thereof with high purity.

### BACKGROUND ART

PEG has been known as a representative hydrophilic macromolecule to form hydrogen bonds with water molecules, together with natural macromolecules and synthetic macromolecules.

Further, PEG is soluble in various organic solvents, with little or no human toxicity. Since PEG has a completely elongated configuration in water, conjugation with other medicines (protein, peptide, enzyme, gene, or the like) affords reducing toxicity of the medicine molecules by virtue of steric hindrance, thereby giving protection from immune system. Thus, PEG can be applied to a variety of medicines as a means to lengthen their half-life in human blood plasma.

Furthermore, PEG can be combined with a medicine being difficult to be applied, which has excellent activity but high toxicity and low solubility, to enhance the solubility of PEG-drug and to reduce the toxicity, thereby improving the activity.

In order to incorporate PEG to another medicine, a method of adding various functional groups to the end of PEG chain for combining it with the medicine.

One of the substances which are combined with various medicines to enhance the solubility and activity thereof, is mPEG-ethylmaleimide.

Conventionally, mPEG-ethylmaleimide could be obtained by one of the two processes: The one is a process comprising the stages of isolation and purification from mPEG-ethylmaleamic acid, chlorination or esterification, and then cyclization (Reaction Formula 1); and the other is a process for preparing it from mPEG-amine via cyclization by using MCM (methoxy carbonyl maleimide) (Reaction Formula 2).

The mPEG-ethylmaleimide prepared by the first process has significantly lower purity measured than that of the second process. During the reactions in the first process, decomposition of PEG chain may occur, thereby resulting in the increase in distribution of molecular weight of the final product. According to the second process for preparing mPEG-maleimide, exact reaction end point cannot be anticipated by the means up to the present. Further, owing to the use of diethyl ether in recrystallization, the process may imply human toxicity and danger of explosion when being applied to a commercial process. In addition, because of by-products (mPEG-amide-imide, mPEG maleamic acid), which are difficult to be removed, contained in the final product, the product is not appropriate to be used in a drug delivery system.

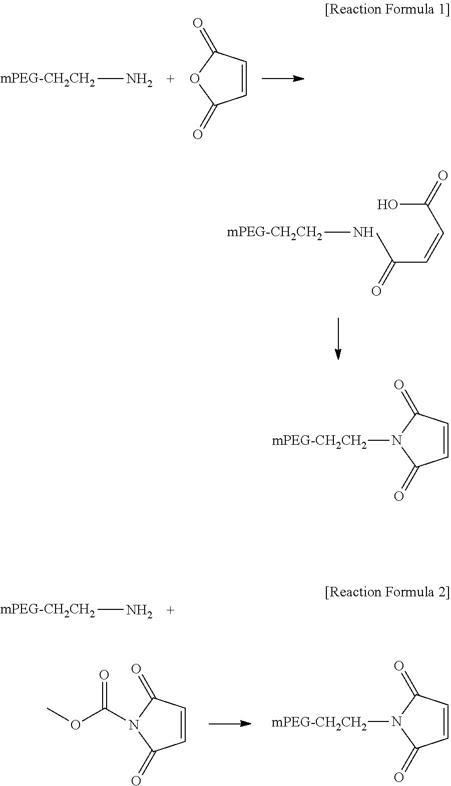

Though U.S. Pat. No. 6,602,498 (Shearwater Corporation) describes the two processes as mentioned above, specific reaction conditions regarding the end point of the reaction (effect of the stirrer rate on the reaction conversion rate, and analytic process); crystallization process that is applicable to economic commercialization; and types of by-products that may be contained in the final product were not stated in detail.

According to U.S. Pat. No. 6,875,841 (NOF Corporation), methoxy polyethyleneglycol (mPEG) was subjected to cyanation and amination to prepare mPEG-propylamine, which was then reacted with maleic anhydride to obtain mPEG-propylmaleamic acid. The mPEG-propylmaleiamic acid thus obtained was subjected to ring formation under the condition of acetic anhydride/acetic acid to prepare mPEG-propylmaleimide. The mPEG-propylmaleimide thus prepared comprises three carbons between the PEG backbone and maleimide in the structure, thus being different from the structure of mPEG-ethylmaleimide. Further, the process comprises complicated reaction stages with four (4) stages from mPEG (cyanation→amination→preparation of maleamic acid→incorporation of maleimide ring). Since the process involves a condition of high pressure and high temperature (>4 MPa, >130° C.), purity of the final product is lower than that of other processes because of decomposition of PEG chain occurred.

According to U.S. Pat. No. 6,828,401 (SunBio Inc.), mPEG-ethylmaleamic acid is firstly prepared, and then mPEG-ethylmaleimide is prepared under condition of diisopropylethylamine/pentafluorophenyl trifluoroacetate/DMF. However, the process gives disadvantages of commercial limitation due to the use of expensive reactants, and much impurities produced.

### DISCLOSURE

### Technical Problem

In order to overcome the problems described above, the present invention provides a process for preparing mPEG-ethylmaleimide at a high yield.

Another subject of the invention is to provide novel process for preparing mPEG-ethylmaleimide with minimum production of intermediates or impurities.

Still another subject of the invention is to provide a process for preparing mPEG-ethylmaleimide with high purity by developing a method to minimize the amount of hydrolysis of mPEG-ethylmaleimide to be converted to mPEG-ethylmaleamic acid during the progress of the reaction.

Still another subject of the invention is to provide a process for producing mPEG-ethylmaleimide which contains not more than 10 mol % of the reaction intermediate compound and not more than 10 mol % of mPEG-ethylmaleamic acid as the by-product produced by hydrolysis of the target compound.

In addition, the present invention provides an effective extraction and crystallization process for the produced mPEG-ethylmaleimide from the reaction medium.

### Technical Solution

The present invention relates to the stage of preparing mPEG-ethylmaleimide with high purity via reacting mPEG-ethylamine with N-methoxycarbonylmaleimide.

The invention provides a process for preparing mPEG-ethylmaleimide which minimizes the content of mPEG-amide-imide as an intermediate and mPEG-ethylmaleamic acid as the by-product, which is characterized by finding the reaction end point by means of NMR during the course of the reaction.

The invention also provides a novel process for preparing mPEG-ethylmaleimide wherein the content of mPEG-ethylmaleamic acid, which is a by-product produced by hydrolysis of the mPEG-ethylmaleimide product, is minimized.

According to another aspect, the invention is characterized by reacting methoxypolyethyleneglycol ethylamine with N-methoxycarbonylmaleimide in an aqueous solution.

According to still another aspect, the invention provides a novel process for preparing mPEG-ethylmaleimide wherein, after determining the reaction end point by means of NMR, the mPEG-ethylmaleimide produced is extracted by phase separation or crystallized by using a commercially available solvent.

The invention also provides a novel process for preparing mPEG-ethylmaleimide wherein the extraction extent is determined by using PAA (polyacrylic acid) to confirm the extent of phase separation during the stage of phase separation.

According to the conventional process for preparing mPEG-ethylmaleimide, too much by-products were produced to effectively obtain the product with purity. This is because the product, mPEG-maleimide is hydrolyzed in the aqueous solution, before the intermediate, mPEG-amide-imide thoroughly converted to the product, so that the product mPEG-ethylmaleimide is converted to mPEG-ethylmaleamic acid, thereby causing the increase of by-products. Thus, the present inventors found that, in order to prepare mPEG-ethylmaleimide with high purity, conversion rate of the intermediate to the product should be increased, while minimizing the conversion of the product to mPEG-ethylmaleamic acid via hydrolysis, and that a method to determine the reaction end point is very important in preparing mPEG-ethylmaleimide with high purity; and completed the present invention.

The reaction mechanism of the present invention is construed as proceeded as follows: Commercially available mPEG-OCH₂CH₂NH₂ of high purity is reacted with N-methoxycarbonylmaleimide at a low temperature of 0˜10° C., preferably at 0˜5° C., and the reaction mixture is extracted by using a halogenated hydrocarbon such as methylene chloride or a hydrocarbon solvent, preferably by using methylene chloride, to prepare mPEG-ethylmaleimide with high purity. In order to minimize production of the intermediate and by-product, conversion rate is measured by using NMR during the reaction. The intermediate produced in the early stage of the reaction (mPEG-amide-imide) has two characteristic peaks (doublet at 6.37 ppm; and doublet at 6.18 ppm). As the reaction time goes by, the area of the characteristic peak (singlet at 6.71 ppm) of mPEG-ethylmaleimide with high purity increases. From the time when most of the characteristic peaks of the intermediate disappear, hydrolysis rapidly proceeds, and the area of characteristic peaks (doublet at 6.31 ppm and doublet at 6.48 ppm) of the by-product produced (mPEG-ethylmaleamic acid) begins to increase. The reaction is completed when the area of the characteristic peak of the intermediate and that of the by-product becomes not more than 10 mol % on the basis of the area of the characteristic peak (singlet at 6.71 ppm) of mPEG-ethylmaleimide with high purity.

NMR analysis is carried out at a low temperature (−10˜5° C.) while stirring is stopped in the reactor. Surprisingly, the reaction does not proceed when stirring is stopped, and thus, the analysis is preferably carried out without separate stirring during the analysis.

The present invention is characterized by preparing mPEG-ethylmaleimide with high purity, which has the end group activity of at least 80%, preferably 80˜99.99% measured by NMR, having molecular weight range of polyethyleneglycol unit of 350˜100,000 and the molecular weight distribution of not more than 1.05.

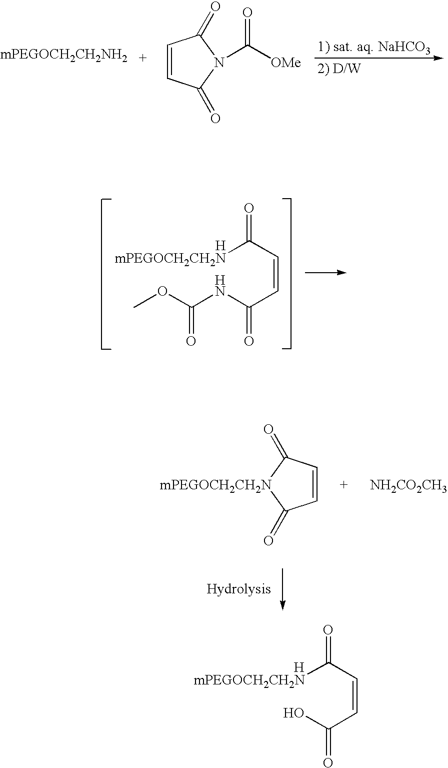

During the process according to the invention, various pollutions by microorganisms originated from human bodies or air may exist. This pollution may cause toxicity if there is endotoxin contained, when the product of the invention, mPEG-ethylmaleimide is conjugated with other medicines (protein, peptide, enzyme, gene, or the like). Thus, a stage to exclude endotoxin from the process for preparing mPEG-ethylmaleimide is required. Accordingly, the stage of removing endotoxin is incorporated by using charcoal to produce product of safety.

The preparation stages are now specifically described.

1) In a reactor purged with nitrogen at ambient temperature, charged are NaHCO₃ and deionized water (D/W), and the temperature inside the reactor is adjusted to 0˜10° C., preferably to 0˜5° C. To the reactor, mPEG-OCH₂CH₂NH₂ having the molecular weight of 100˜100,000 is charged as raw material.

2) Then, N-methoxy carbonyl maleimide is charged into the reactor in an amount of 0.9˜10 equivalents, preferably 1˜5 equivalents on the basis of 1 equivalent of mPEG-OCH₂CH₂NH₂. The mixture is stirred for 0.5˜1 hour, while controlling the stirring rate. In order to accelerate the reaction progress, D/W (50˜55 kg) chilled to 0˜3° C. is additionally incorporated. The reaction is proceeded while confirming the reaction conversion rate every hour by means of NMR. Since mPEG-ethylmaleimide produced may be hydrolyzed to be converted to mPEG-ethylmaleamic acid when the reaction time is prolonged, control of the reaction duration is very important. Unexpectedly, the present inventors found very remarkable difference in the reaction rate between the reactants stirred in the reactor and the same without stirring. They also found the fact that the reaction does not significantly proceed if not with stirring, so that the end point can be controlled by analysis by means of ¹H-NMR with sampling of the reactant while the stirring is stopped during the reaction, without any special reaction during the analysis.

mPEG-amide-imide (500 MHz, ¹H-NMR): (d, 6.37 ppm), (d, 6.18 ppm)

mPEG-ethylmaleimide: (s, 6.71 ppm)

mPEG-ethylmaleamic acid: (d, 6.31 ppm), (d, 6.48 ppm)

The content is calculated as follows:

mPEG-ethylmaleimide (500 MHz, ¹H-NMR): With the standard value 3 for the area of the characteristic peak of methoxy group at 3.29 ppm, the area of the characteristic peaks at 6.20, 6.32, 6.71 ppm are calculated.

$$\begin{matrix}mPEG \text{-} ethylmaleimide ( \% ) = \frac{Area of vinyl proton ( 2 H ) at 6.7 ppm}{2} \times 100. & 1 \\ mPEG \text{-} Amide \text{-} Imide ( \% ) = \frac{Area of 6.20 ppm}{\begin{matrix}( Area of 6.20 ppm ) + ( \frac{Area of 6.71 ppm}{2} ) + \\ ( Area of 6.32 ppm )\end{matrix}} \times 100. & 2 \\ mPEG \text{-} Maleamic acid ( \% ) = \frac{Area of 6.32 ppm}{\begin{matrix}( Area of 6.20 ppm ) + ( \frac{Area of 6.71 ppm}{2} ) + \\ ( Area of 6.32 ppm )\end{matrix}} \times 100. & 3\end{matrix}$$

3) When the end point is determined, incorporated is an organic solvent selected from hydrocarbons or halogenated hydrocarbons such as pentane, hexane, heptane, octane, methylene chloride and chloroform, and the product is extracted in the organic layer from the aqueous layer. Whether the mPEG type reactant, the intermediate, the product and the by-product are extracted into the organic layer from the aqueous layer or not is confirmed by whether emulsification occurs with incorporation of aqueous polyacrylic acid solution to the aqueous layer. Thus, additional incorporation of organic solvent or methanol is employed as a means to promote the phase separation. As the additional solvent to improve the extraction efficiency by promoting the phase separation, methanol is preferably and efficiently used. The aqueous polyacrylic acid solution is preferably used after incorporating some portion of hydrochloric acid to the aqueous polymer solution of 1000˜30000 cP rather than being used alone, so that the progress of extraction can be exactly seen. For example, polyacrylic acid (Wako, 25% aqueous solution, 8,000˜12,000 cP (30° C.)) may be used as a mixture with conc. HCl (10 ml) and H₂O (105 ml) after being shaken for 30 minutes.

4) Then, the isolated organic layer is washed by adding an equivalent amount of water. Stage 3) can be repeatedly carried out if required.

5) PAA test is performed. If PEG is not detected from the aqueous layer any more, phase separation is carried out. The MC layer is dried over MgSO₄ (6 kg), and filtered to recover the product solution.

6) Then, the organic layer is concentrated, and IPA/heptane (about 1:2 v/v) previously cooled to −5˜0° C. is incorporated thereto to crystallize the product. As the crystallization solvent, MTBE, IPA and heptane may be used alone, or in a combination of two or more solvents.

7) To the wet cake obtained, MC is incorporated, and the solid is completely dissolved. Then, charcoal is added and the mixture stirred to minimize the content of endotoxin (standard: 2 EU/g, test method: USP 24 <85> Bacterial Endotoxins Test). The target compound has excellent solubility in MC solvent, and endotoxin is adsorbed on charcoal, so that most of the target compound can be recovered. Then, charcoal is removed by using a filter filled with Celite microparticles, and stage 6) is repeated to crystallize the product. The final product is obtained after filteration and drying.

### BEST MODE

### Examples

Examples are described for more specific explanation of the present invention, but the invention is not limited to those Examples. Percentage described herein means mol % unless otherwise specified.

### Example 1

To a 300 L reactor purged with nitrogen at ambient temperature, charged were NaHCO₃ (5.13 kg) and D/W (56 kg). After cooling the temperature inside the reactor to 0˜1° C., mPEG-NH₂ (6 kg) having molecular weight of 5000 was charged and dissolved therein. Then, N-methoxycarbonylmaleimide (0.94 kg) was incorporated thereto, and the mixture was stirred at 50 rpm for 1 hour. Additional D/W (52 kg) was incorporated and the reaction proceeded with confirming the reaction conversion rate every hour by using NMR.

| Reaction | Target compound | Intermediate | By-product |
|---|---|---|---|
| duration (hr) | (mol %) | (amide-imide, %) | (Maleamic acid, %) |
| 1 | 24.25 | 75.75 | ND |
| 2 | 72.82 | 27.18 | ND |
| 3 | 76.32 | 23.68 | Trace |
| 4 | 76.42 | 19.59 | 3.99 |
| 4.5 | 87.19 | 7.36 | 5.45 |

After the reaction completed (4.5 hr), methylene chloride (55 L) was charged, and the mixture was thoroughly stirred and extracted.

In order to confirm whether all of the product was extracted from the aqueous layer, PAA [prepared by mixing polyacrylic acid (Wako, 25%, 8,000-12,000 cP (30° C.), 5 ml), conc. HCl (10 ml) and H₂O (105 ml) and shaking the mixture for 30 minutes] solution (0.5 ml) was incorporated to 50 ml of the aqueous layer, and existence of residual mPEG-Mal (5 k) was confirmed in the aqueous layer. No suspension occurred, and thus it was confirmed that the product was totally extracted with methylene chloride into organic phase. When the isolated MC layer was washed with D/W (55 L), emulsification and suspension occurred in the aqueous layer. Methanol (12 L) was additionally incorporated as a dispersing agent to induce complete phase separation. Then, by PAA test, it was confirmed that no more PEG derivative is detected in the aqueous layer. Phase separation was carried out and MgSO₄ (6 kg) was incorporated to the MC layer, and the mixture was stirred and filtered. After concentrating under reduced pressure to make the total volume of the organic layer to 10 L, the product was added dropwise to the solution of IPA/heptane (21/41 kg) previously cooled to −5˜0° C., and the mixture was stirred to crystallize the product. To the white crystalline wet cake obtained after filtration, 50 L of MC was incorporated again. After complete dissolution, charcoal (1 kg) was incorporated thereto, and the mixture was stirred for 30 minutes to minimize endotoxin content. Charcoal was removed by using Celite, and the solution was concentrated under reduced pressure to give 10 L of total volume. The solution was added dropwise to IPA/heptane (21/41 kg), and the resultant mixture was stirred and crystallized. Final product was obtained by filtration and drying. Yield: 95 mol %. The product was confirmed by ¹H-NMR [(500 MHz, CDCl₃): —CH═CH— 6.71 ppm, PEG backbone 3.45-3.8 ppm, —OCH₃ 3.29 ppm], and characterized by following properties:

Endotoxin (standard: <2.0 EU/g): 0.25, mPEG ethylenemaleimide (by 1H-NMR): 87.19%, mPEG-amide-imide (by NMR): 7.36%, mPEG-maleamic acid (by NMR): 5.45%.

### Example 2

Same procedure as described in Example 1 was repeated but the stirring rate was 80 rpm instead of 50 rpm, and the reaction duration was 2 hours. The results are shown below:

Yield (93 mol %)

Endotoxin (standard: <2.0 EU/g): 0.5

mPEG ethylenemaleimide (by ¹H-NMR) (86.7%), mPEG amide-imide (by NMR) (6.3%), mPEG maleamic acid (by NMR) (7.0%)

### Example 3

Same procedure as described in Example 1 was repeated but the stirring rate was 100 rpm instead of 50 rpm, and the reaction duration was 4.5 hours. The results are shown below:

Yield (92 mol %),

Endotoxin (standard: <2.0 EU/g): 0.5

mPEG ethylenemaleimide (by ¹H-NMR) (82.2%), mPEG amide-imide (by NMR) (4.1%), mPEG maleamic acid (by NMR) (13.7%)

### Example 4

Same procedure as described in Example 1 was repeated but the stirring rate was 150 rpm instead of 50 rpm, and the reaction duration was 6.5 hours. The results are shown below:

Yield (94 mol %),

Endotoxin (standard: <2.0 EU/g): 0.25

mPEG ethylenemaleimide (by ¹H-NMR) (78.3%), mPEG amide-imide (by NMR) (N.D.), mPEG maleamic acid (by NMR) (21.2%)

### INDUSTRIAL APPLICABILITY

As described above, mPEG-ethylenemaleimide with high purity (at least 80%) can be prepared according to the process of the invention with maintaining the content of the intermediate and that of the by-product as low as not more than 10%, respectively.

1. A process for preparing methoxypolyethyleneglycol ethylmaleimide, comprising the stages of
1) reacting methoxypolyethyleneglycol ethylamine with N-methoxycarbonylmaleimide in an aqueous solution in the presence of a base;
2) determining the end point of the product during the reaction via NMR measurement;
3) extracting the product by phase separation using organic solvent, after completion of the reaction; and
4) concentrating the organic phase extracted and recrystallizing methoxypolyethyleneglycol ethylmaleimide therefrom.

2. A process for preparing methoxypolyethyleneglycol ethylmaleimide according to claim 1, wherein the reaction temperature is 0° C. to 10° C.

3. A process for preparing methoxypolyethyleneglycol ethylmaleimide according to claim 1, wherein the recrystallization is carried out by using a mixed solvent of isopropanol and heptane.

4. A process for preparing methoxypolyethyleneglycol ethylmaleimide according to claim 1, wherein the purity of methoxypolyethyleneglycol maleimide is 80% or higher.

5. A process for preparing methoxypolyethyleneglycol ethylmaleimide according to claim 4, characterized in that the purity is obtained by a process for determining the reaction end point via NMR measurement where the area of characteristic peak of mPEG-amide-imide as the intermediate and that of mPEG-ethylmaleamic acid as the hydrolytic product are not more than 10 mol %, respectively, on the basis of the area of the characteristic peak of methoxypolyethyleneglycol ethylmaleimide.

6. A process for preparing methoxypolyethyleneglycol ethylmaleimide according to claim 1, which further comprises the stage of removing endotoxin from the recrystallized product by incorporating charcoal.

7. A process for preparing methoxypolyethyleneglycol ethylmaleimide according to claim 6, which further comprises the stage of dissolving the recrystallized product in methylene chloride before incorporating charcoal.

8. A process for preparing methoxypolyethyleneglycol ethylmaleimide according to claim 1, which further comprises the stage of determining whether additional extraction via phase separation has to be carried out or not, via PAA test stage during the extraction.

9. A process for preparing methoxypolyethyleneglycol ethylmaleimide according to claim 8, wherein methanol is incorporated to facilitate phase separation when the additional extraction is necessary according to the PAA test.

10. A process for preparing methoxypolyethyleneglycol ethylmaleimide according claim 1, wherein said NMR measurement is a process for determining the reaction end point where the area of characteristic peak of mPEG-amide-imide as the intermediate and that of mPEG-ethylmaleamic acid as the hydrolytic product are not more than 10 mol %, respectively, on the basis of the area of the characteristic peak of methoxypolyethyleneglycol ethylmaleimide.

11. A process for preparing methoxypolyethyleneglycol ethylmaleimide according to claim 10, wherein the molecular weight of said methoxypolyethyleneglycol ethylmaleimide is 350 to 100,000.
